(19) United States
(12) Patent Application Publication
(10) Pub. No.: US 2006/0205724 A1
(43) Pub. Date: Sep. 14, 2006

(54) Antagonizing nk-1 receptors inhibits consumption of substances of abuse

(75) Inventors: Olive Foster, Charleston, SC (US); Jennifer Whistler, El Cerrito, CA (US)

Correspondence Address: QUINE INTELLECTUAL PROPERTY LAW GROUP, P.C., P O BOX 458, ALAMEDA, CA, 94501, US

(73) Assignee: The Regents of the University of California

(21) Appl. No.: 10/520,053

(22) PCT Filed: May 29, 2003
(86) PCT No.: PCT/US03/17181

§ 371 Date: Feb. 27, 2006

Related U.S. Application Data
(60) Provisional application No. 60/384,561, filed on May 29, 2002.

(51) Int. Cl. A61K 31/5377

(52) U.S. Cl. 514/235.5

(57) ABSTRACT

This invention pertains to the surprising discovery that administration of one or more NK1 receptor antagonists to a mammal can inhibit self-administration of a substance of abuse (e.g. alcohol). In one embodiment, this invention provides a method of inhibiting or reducing self-administration of a substance of abuse by a mammal. The method involves administering to the mammal an NK1 receptor antagonist in a concentration sufficient to reduce self-administration of a substance of abuse and/or craving for a substance of abuse (e.g. ethanol).

### CROSS-REFERENCE TO RELATED APPLICATIONS

This application claims priority to and benefit of U.S. Ser. No. 60/384,561, filed on May 29, 2002, which is incorporated herein by reference in its entirety for all purposes.

### STATEMENT AS TO RIGHTS TO INVENTIONS MADE UNDER FEDERALLY SPONSORED RESEARCH AND DEVELOPMENT [Not Applicable ]

### FIELD OF THE INVENTION

This invention pertains to the fields of neurobiology and behavior. In particular, this invention pertains to the discovery that inhibition of an NK1 receptor reduces or eliminates chronic consumption of substances of abuse.

### BACKGROUND OF THE INVENTION

Chronic consumption of substances of abuse (e.g. alcohol abuse and alcohol dependence, i.e., alcoholism) are serious public health problems of modern society. According to U.S. Government projections from studies conducted in the mid-1980s, in the United States alone, an estimated 13 million adults exhibit symptoms of alcohol dependence due to excessive alcohol intake, and an additional 7 million abuse alcohol without showing symptoms of dependence. Alcohol dependence and abuse are very expensive: in economic and medical terms. It is estimated that alcohol abuse alone will costs the U.S. well over $200 billion/year. The social and psychological damages inflicted on individuals as a consequence of alcohol abuse, e.g., children born with fetal alcohol syndrome (FAS) and victims of alcohol-related accidental death, homicide, suicide, etc., are immense.

While it is generally accepted that chronic consumption of substances of abuse presents staggering international economic, social, medical, and psychological repercussions, success in preventing or otherwise ameliorating the consequences of these problems has been an elusive goal. Only very recently the public view that consumption of substances of abuse is remediable solely by moral imperatives has been changed to include an awareness of substance dependence as a physiological aberration whose etiology may be understood and for which therapy may be found through scientific pursuits.

Recent studies of substance abuse have been based on the hypothesis that such-abuse can be understood and dealt with at the molecular level. Such a molecular understanding, if achieved, would provide a basis for the identification and development of appropriate therapeutic agents. Where clinical manifestations of substance abuse (e.g. alcoholism and alcohol abuse) are the consequence of aberrations or defects within one or more metabolic pathways, such conditions may be effectively treated by the use of appropriate pharmaceuticals.

### SUMMARY OF THE INVENTION

This invention pertains to the surprising discovery that administration of one or more NK1 receptor antagonists to a mammal can inhibit self-administration of a substance of abuse (e.g. alcohol). In one embodiment, this invention provides a method of inhibiting or reducing self-administration of a substance of abuse by a mammal. The method involves administering to the mammal an NK1 receptor antagonist in a concentration sufficient to reduce self-administration of a substance of abuse and/or craving for a substance of abuse (e.g. ethanol).

In another embodiment, this invention provides a kit for inhibiting or reducing self-administration of a substance of abuse. The kit comprises a container containing one or more NK1 receptor antagonists (e.g., LY303870, Sigma WIN 51,708, GR205171A, Takeda TAK-637, SR-140333, Merck MK869, MEN 11467, triazole NK1 receptor antagonists (see WO 99/38533A1),.CP-99,994; GlaxoSmith-Kline GW597599, CJ-12,255, etc.) and instructional materials teaching the use of an NK1 receptor antagonist to inhibit self-administration of a substance of abuse.

In still another embodiment, this invention provides a method of screening for an agent that reduces self-administration of a substance of abuse by a mammal. The method involves contacting a cell comprising an NK1 receptor with a test agent; and detecting expression or activity of said NK1 receptor or an NK1 receptor pathway, where an inhibition of NK1 receptor expression or activity as compared to NK1 receptor expression or activity in a control cell indicates that said test agent is a candidate agent for use in reducing self-administration of a substance of abuse by a mammal.

### DEFINITIONS

The term “substance of abuse” refers to a substance that is psychoactive and that induces tolerance and/or addiction. Substances of abuse include, but are not limited to stimulants (e.g. cocaine, amphetamines), opiates (e.g. morphine, heroin), cannabinoids (e.g. marijuana, hashish), nicotine, alcohol, substances that mediate agonist activity at the dopamine D2 receptor, and the like. Substances of abuse include, but are not limited to addictive drugs.

An “NK1 receptor”, an “NK1 receptor”, or an “NK₁” receptor refer to a tachykinin NK1-receptor.

An “NK1” receptor antagonist” refers to a substance that directly or indirectly reduces or blocks activity mediated by an NK1 receptor in response to the cognate ligand of that receptor. The inhibition can be through direct action at the receptor, and/or through action at one or more genes controlling expression of the receptor, and/or through one or more components of a-pathway activated by agonistic activity of an NK1 receptor.

By “prior exposure” to ethanol or other substances of abuse (e.g., addictive drugs) is meant that a sample has been exposed to exogenous ethanol, or other substances of abuse, before a particular point in time, such as, for example, before testing for such exposure. Usually the sample has been exposed at most two weeks before testing, preferably less than a week, even more preferably within 48 hours before testing. An example of prior exposure is found in a sample obtained from a mammal that has a detectable blood alcohol level. However, the exposure need not be continuous and it need not occur immediately before testing. For example, many alcoholics have blood alcohol levels close to or equal to zero in the morning. Thus, the phrase “prior exposure” includes chronic and/or episodic exposure.

By “chronic exposure” to ethanol or other substances of abuse (e.g. addictive drugs) is meant that a sample or an organism has been exposed to exogenous ethanol or other substances of abuse chronically before a particular point in time. The sample might not have been exposed immediately before testing is performed, or even within 48 hours before testing, but it has been exposed on a recurrent or prolonged basis for a time sufficient for the cellular effects of such exposure to be detectable whether or not the substance of abuse (e.g. addictive drug) is present in the sample at a detectable level. An example of chronic exposure is found in a sample obtained from a mammal that has been chronically consuming alcohol whether or not the mammal has a detectable blood alcohol level at the time the sample is obtained.

### BRIEF DESCRIPTION OF THE DRAWINGS

FIG. 1 illustrates the effect of an NK1 receptor antagonist (LY303870) on the loss of righting reflex in rats administrated ethanol at 3 and 4 g/kg. *P<0.05 vs vehicle-treated animals. n=9-10 per group.

FIG. 2 shows the effect of LY303870 on self-administration of ethanol by a rat. The graphs show ethanol intake, water intake, and ethanol preference. LY303870 shows a significant effect on ethanol consumption and preference.

FIG. 3 shows the effect of WIN 51,708 on self-administration of ethanol by a rat. The graphs show ethanol intake, water intake, and ethanol preference.

### DETAILED DESCRIPTION

This invention pertains to the surprising discovery that administration of one or more NK1 receptor antagonists to a mammal can inhibit (reduce) self-administration of a substance of abuse (e.g. alcohol). It is believed that this effect can be realized in a mammal presently engaged in chronic consumption of the substance of abuse, or in a mammal that has stopped/withdrawn from such chronic consumption. In the latter case, it is believed that administration of NK1 receptor antagonists reduces cravings for the substance and consequently reduces the likelihood of a recurrence of such consumption (relapse).

In the former case (where the mammal is engaged in chronic consumption of a substance of abuse) it is believed that administration of the NK1 antagonist will reduce self-administration of the substance of abuse (e.g. reduce craving) and facilitate recovery from a pattern of such chronic consumption.

It is noted that the data indicate that administration of an NK1 antagonist reduces preference for a substance of abuse. Thus, it appears that the reduced consumptive behavior does not simply reflect a general diminution of drinking behavior.

In view of this, it is believed that NK1 receptor antagonists can be administered to a subject (e.g. a human, a non-human mammal, etc.) to reduce self administration of a substance of abuse. (e.g. alcohol). The subject can be one that is presently engaged in chronic consumption of the substance of abuse, or one that has stopped/withdrawn from such chronic consumption

I. NK1 Receptor Antagonists.

Numerous NK1 receptor antagonists are suitable for practice of the methods of this invention, are well known to those of skill in the art, and are in clinical trials (e.g. for treatment of depression and/or for the treatment of migraine headaches).

In certain embodiments, the NK1 receptor antagonists need not be specific for the NK1 receptor. Thus, for example, it is acceptable for certain antagonists to also inhibit other receptors such as the NK2 and/or the NK3 receptors as well as the NK1 receptor. In preferred embodiments, the NK1 receptor antagonists are NK1 receptor specific antagonists that preferentially inhibit NK1 receptors as compared to NK2 and/or NK3 receptors and/or other receptors associated with the tachykinin pathway(s). Preferred NK1 receptor-specific antagonists show at least a detectable preference for NK1 inhibition as compared to NK2 or NK3, preferably at least a 1.5 fold greater inhibition of NK1 as compared to NK2 and/or NK3 at the same concentration, more preferably at least a 2 fold greater inhibition of NK1 as compared to NK2 and/or NK3 at the same concentration, still more preferably at least a 1.5 fold greater inhibition of NK1 as compared to NK2 and/or NK3 at the same-concentration, and most preferably at least a 10 fold, 20 fold, 50 fold, or 100 fold greater inhibition of NK1 as compared to NK2 and/or NK3 at the same concentration.

In various embodiments, the NK1 receptor antagonists include, but are not limited to acyclic compounds (see, e.g., U.S. Pat. No. 5,387,595), naphthyl compounds (see, e.g., U.S. Pat. No. 5,491,140), morpholine and thiomorpholine derivatives (see, e.g., U.S. Pat. Nos. 5,512,570, 5,637,699, 5,691,336, 5,716,942, 5,719,147, 5,719,149, 5,747,491, 5,780,467, 5,824,678, 5,872,116, 5,922,706, 5,968,934, 5,985,896, 6,048,859, 5,985,896, 5,891,875, and the like), piperidines (see, e.g., U.S. Pat. Nos. 5,530,009, 5,610,165, 5,985,896, 6,096,766, and the like), azacyclic compounds (see, e.g., U.S. Pat. Nos. 5,633,266, 5,665,883, 5,760,018, and the like), spiroethercycloalkyl compounds (see, e.g., U.S. Pat. Nos. 5,877,191, 5,929,094, and the like), various a benzo[b]thiophenes (see, e.g., U.S. Pat. Nos. 5,492,927, 5,525,624, and 5,670,499), triazole derivatives (see, e.g., U.S. Pat. No. 5,710,161), benzylaminoquinuclidines (see, e.g., U.S. Pat. Nos. 5,869,499 and 5,741,797), bisindoles (see, e.g., U.S. Pat. No. 5,792,760), 2-acylaminopropanamides (see, e.g., U.S. Pat. No. 5,565,568), pyrrolidinyl compounds (see, e.g., U.S. Pat. No. 5,607,947), cycloalkyl compounds (see, e.g., U.S. Pat. No. 5,750,549), quaternary ammonium compounds (see, e.g., U.S. Pat. Nos. 6,207,678 and 6,380,396), hexamethyleneiminyl compounds (see, e.g., U.S. Pat. No. 5,563,133), and the like.

NK1 receptor antagonists are also described in published European Patent, Application Nos. EP 0 360 390, EP 0 394 989, EP 0 429 366, EP 0 443 132, EP 0 482 539, EP 0 512 901, EP 0 512 902, EP 0 514 273, EP 0 514 275, EP 0 517 589, EP 0 520 555, EP 0 522 808, EP 0 528 495, EP 0 532 456, EP 0 533 280, EP 0 536 817, EP 0 545 478, EP 0 577 394, EP 0 590 152, EP 0599 538, EP 0 610 793, EP 0634402, EP 0 686 629, EP 0 693 489, EP 0 694 535, EP 0 699 655, EP 0 699 674, EP 0 707 006, EP 0 708 101, EP 0 714 891, EP 0 723 959, EP 0 733 632 and EP 0 776 893; and in International Patent Application Nos. WO 90/05525, WO 90/05729, WO 91/09844, WO 91/18899, WO 92/01688, WO 92/06079, WO 92/12151, WO 92/15585, WO 92/17449, WO 92/20661, WO 92/20676, WO 92/21677, WO 93/00330, WO 93/00331, WO 93/01159, WO 93/01165, WO 93/01169, WO 93/01170, WO 93/06099, WO 93/09116, WO 93/10073, WO 93/14113, WO 93/18023, WO 93/19064, WO 93/21155, WO 93/21181, WO 93/23380, WO 93/24465, WO 94/01402, WO 94/02461, WO 94/03429, WO 94/03445, WO 94/04494, WO 94/04496, WO 94/05625, WO 94/07843, WO 94/10165, WO 94/10167, WO 94/10168, WO 94/10170, WO 94/11368, WO 94/13639, WO 94/13663, WO 94/14767, WO 94/15903, WO 94/19320, WO 94/19323, WO 94/20500, WO 94/26735, WO 94/26740, WO 94/29309, WO 95/02595, WO 95/04040, WO 95/04042, WO 95/06645, WO 95/07886, WO 95/07908, WO 95/08549, WO 95/11880, WO 95114017, WO 95/15311, WO 95/16679, WO 95/17382, WO 95/18124, WO 95/18129, WO 95/19344, WO 95/20575, WO 95/21819, WO 96/22525, WO 95/23798, WO 95/26338, WO 95/28418, WO 95/30674, WO 95/30687, WO 96/05193, WO 96/05203, WO 96/06094, WO. 96/07649, WO 96/10562, WO 96/16939, WO 96/18643, WO 96/20197, WO 96/21661, WO 96/29304, WO 96/29317, WO 96/29326, WO 96/29328, WO 96/31214, WO 96/32385, WO 96/37489, WO 97/01553, WO 97/01554, WO 97/03066, WO 97/08144, WO 97/14671, WO 97/17362, WO 97/18206, WO 97/19084, WO 97/19942 and 97/21702; and in British-Patent Applications Nos. 2 266 529, 2 268 931, 2 269 170, 2 269 590, 2 271 774, 2 292 144, 2 293 168, 2 293 169, and 2 302 689.

Various NK1 receptor antagonists particularly useful in the present invention include, but are not limited to various morpholine, and other derivatives including, but not limited to: 2-(S)-(3,5-bis(trifluoromethyl)benzyloxy)-3(S)-(4-fluorophenyl)-4-(3-(5-oxo-1H,4H-1,2,4-triazolo)methyl)morpholine; 2-(R)-(1-(R)-(3,5-bis(trifluoromethyl)phenyl)ethoxy)-4-(3-(5-oxo-1H,4H-1,2,4-triazolo)methyl)-3-(S)-phenyl-morpholine; 2-(S)-(3,5-bis(trifluoromethyl)benzyloxy)-4-(3-(5-oxo-1H,4H-1,2,4-3-(S)-phenyl-morpholine; 2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl)morpholine; 2-(R)-(1-(R)-(3,5-bis(trifluoromethyl)phenyl)ethoxy)-4-(5-(N,N-dimethylamin o)methyl-1,2,3-triazol-4-yl)methyl-3-(S)-phenylmorpholine; 2-(R)-(1-(R)-(3,5-bis(trifluoromethyl)phenyl)ethoxy)-4-(5-(N,N-dimethylamin o)methyl-1,2,3-triazol4-yl)methyl-3-(S)-(4-fluorophenyl)morpholine; 2-(R)-(1-(R)-(3,5-bis(trifluoromethyl)phenyl)ethoxy)-3-(S)-(4-fluorophenyl)-4-(3-(4-monophosphoryl-5-oxo-1H-1,2,4-triazolo)methyl)morpholine; 2-(R)-(1-(R)-(3,5-bis(trifluoromethyl)phenyl)ethoxy)-3-(S)-(4-fluorophenyl)-4-(3-(1-monophosphoryl-5-oxo-1H-1,2,4-triazolo)methyl)morpholine; 2-(R)-(1-(R)-(3,5-bis(trifluoromethyl)phenyl)ethoxy)-3-(S)-(4-fluorophenyl)-4-(3-(2-monophosphoryl-5-oxo-1H-1,2,4-triazolo)methyl)morpholine; 2-(R)-(1-(R)-(3,5-bis(trifluoromethyl)phenyl)ethoxy)-3-(S)-(4-fluorophenyl)-4-(3-(5-oxyphosphoryl-1H-1,2,4-triazolo)methyl)morpholine; 2-(S)-(1-(R)-(3,5-bis(trifluoromethyl)phenyl)ethoxy)-3-(S)-(4-fluorophenyl)-4-(3-(1-monophosphoryl-5-oxo-4H-1,2,4-triazolo)methyl)morpholine; 2-(R)-(1-(R)-(3,5-bis(trifluoromethyl)phenyl)ethoxy)-4-(4-N,N-dimethylamino but-2-yn-yl)-3-(S)-(4-fluorophenyl)morpholine; (3S,5R,6S)-3-[2-cyclopropoxy-5-(trifluoromethoxy)phenyl-1-oxa-7-aza-spiro[4.5]decane; and (3R,5R,6S)-3-[2-cyclopropoxy-5-(trifluoromethoxy)phenyl-1-oxa-7-aza-spiro[4.5]decane. The synthesis of such compounds is described in detail in U.S. Pat. No. 5,919,781, European Patent Application No: EP 0 577 394, and International Patent Application Nos. 95/08549, 95/18124, 95/23798, 96/05181, PCT/GB97/01630.

In certain preferred embodiments, various NK1 receptor antagonists include, but are not limited to, LY303870, GR205171A, Takeda TAK-637, SR-140333, Merck MK869, MEN 11467, triazole NK1 receptor antagonists (see WO 99/38533A1), CP-99,994, GlaxoSmith-Kline GW597599, CJ-12,255, and the like. In particularly preferred embodiments, the NK1 receptor antagonist is LY303870 and/or Sigma WIN 51,708. It will be appreciated, that in certain embodiments, two or more different NK1 receptor antagonists are used in combination.

II. Pharmaceutical Formulations.

The NK1 receptor antagonists used in the methods of this invention, (e.g., to inhibit:chronic consumption of a substance of abuse) can be prepared and administered in a wide variety of rectal, oral and parenteral dosage forms for treating and preventing chronic consumption of a substance of abuse (e.g. alcoholism) and/or withdrawal from such chronic consumption, or post-withdrawal cravings and/or recidivism, and the like. One or more NK1 receptor antagonists can be administered by injection, that is, intravenously, intramuscularly, intracutaneously, subcutaneously, intraduodenally, or intraperitoneally. Also, the compounds can be administered by inhalation, for example, intranasally. Additionally, the compounds can be administered transdermally.

The active molecules (e.g., NK1 receptor antagonists) can comprise as the active component, either a compound as a free base, acid, or a corresponding pharmaceutically acceptable salt of such compound. The active compound generally is present in a concentration of about 1% to about 95% by weight of the formulation. The active molecules are typically combined with a pharmaceutically acceptable carrier (excipient) to form a pharmacological composition. Pharmaceutically acceptable carriers can contain a physiologically acceptable compound that acts, for example, to stabilize the composition or to increase or decrease the absorption of the agent. Physiologically acceptable compounds can include, for example, carbohydrates, such as glucose, sucrose, or dextrans, antioxidants such as ascorbic acid or glutathione, chelating agents, low molecular weight proteins, compositions that reduce the clearance or hydrolysis of the anti-mitotic agents, or excipients or other stabilizers and/or buffers.

Other physiologically acceptable compounds include, but are not limited to wetting agents, emulsifying agents, dispersing agents, and/or preservatives that are particularly useful for preventing the growth or action of microorganisms. Various preservatives are well known and include, for example, phenol and ascorbic acid. One skilled in the art would appreciate that the choice of a pharmaceutically acceptable carrier, including a physiologically acceptable compound depends, for example, on the route of administration of the NK1 receptor antagonist(s) and on the particular physio-chemical characteristics of the NK1 receptor antagonists(s).

In various embodiments the NK1 receptor antagonists(s) can be provided in a substantially dry and/or pure form to be combined with a diluent/excipient at the time of use or one or both agents can be provided already combined with an appropriate excipient (e.g. in a unit dosage form). In other embodiments, NK1 receptor antagonists(s) are provided combined in a compatible excipient.

Various preferred pharmaceutically acceptable carriers can be either solid, liquid, semi-solid, or gel. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid which is in a mixture with the finely divided active component (NK1 receptor antagonist). In tablets, the active component is mixed with the carrier having the necessary binding properties in suitable proportions and compacted in the shape and size desired.

The powders and tablets (or other formulations) preferably at least 0.1%, preferably at least 1%, more preferably at least about 5%, and most preferably at least about. 10% of the active compound. The powders and tablets (or other formulations) preferably contain, at most about 98%, more preferably at most 90%, still more preferably at most 80%, and most preferably at most 709% of the active ingredient(s) (NK1 receptor antagonist(s)).

Suitable carriers include, but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound(s) with encapsulating material as a carrier providing a capsule in which the active component; with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogenous mixture is then poured into convenient sized molds, allowed to cool, and thereby to solidify.

Liquid form preparations include solutions, suspensions, and emulsions, for example, water or water propylene glycol solutions. For parenteral injection, liquid preparations can be, formulated in solution in aqueous polyethylene glycol solution.

Aqueous solutions suitable for oral use can be prepared by dissolving the active component in water and adding suitable colorants, flavors, stabilizing, and thickening agents as desired.

Aqueous suspensions suitable for oral use can be made by dispersing the finely divided active component(s) in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers; buffers, artificial and natural sweeteners, dispersants, thickeners, solubilizing agents, and the like.

The pharmaceutical preparation is preferably in unit dosage form. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit-dose preparation may be varied or adjusted, e.g., from 1 to 1000 mg, preferably 10 to 100 mg according to the particular application and the potency of the active component. The composition can, if desired, also contain other compatible therapeutic agents.

The concentration of therapeutic agent in these formulations can vary widely, and will be selected primarily based on fluid volumes, viscosities, body weight and the like in accordance with the particular mode of administration selected and the patient s needs. Actual methods for preparing administrable compositions will be known or apparent to those skilled in the art and are described in more detail in such publications as Remington's Pharmaceutical Science, 15th ed., Mack Publishing Company, Easton, Pa. (1980).

Dosages for typical therapeutics, particularly for NK1 receptor antagonists, are known to those of skill in the art. Moreover, such dosages are typically advisorial in nature and may be adjusted depending on the particular therapeutic context, patient tolerance, etc. Single or multiple administrations of the compositions may be administered depending on the dosage and frequency as required and tolerated by the patient.

In preferred embodiments, the NK1 receptor antagonist(s) utilized in the methods of this invention are administered at a dose that is effective to inhibit (e.g. to reduce or eliminate self-administration of a substance of abuse and/or to reduce the craving for the substance of abuse) (e.g., a statistically significant decrease at the 90%, more preferably at the 95%, and most preferably at the 98% or 99% confidence level). Preferred effective amounts range from about 0.1 mg/kg daily to about 1000 mg/kg daily, more preferably from about 1 mg/kg daily to about 500 mg/kg daily, still more preferably from about 1 mg/kg daily to about 100 or 500 mg/kg daily, and most preferably from about 1 mg/kg daily to about 10, 20, or 50 mg/kg daily. The compounds can also be used prophylactically at the same dose levels.

The dosages, however, may be varied depending upon the requirements of the patient, the severity of the condition being treated, and the compound being employed. Determination of the proper dosage for a particular situation is within the skill of the art. Generally, treatment is initiated with smaller dosages which are less than the optimum dose of the compound. Thereafter, the dosage is increased by small increments until the optimum effect under the circumstance is reached. For convenience, the total daily dosage may be divided and administered in portions during the day if desired. Typical dosages will be from about 0.1 to about 500 mg/kg, and ideally about 1 to about 50 mg/kg.

III. Kits.

In another embodiment, this invention provides therapeutic kits for practice of the methods of this invention: Such kits preferably include a container containing one or more NK1 receptor antagonists. The NK1 receptor antagonists can be formulated in combination with a pharmaceutically acceptable excipient and/or in a unit dosage form.

The kit can comprise packaging that retains and presents the: medicaments (NK1 receptor antagonists) at-separate respective consecutive locations identified by visibly discernible indicia and the times at which the medicaments are to be taken by the patient. In various embodiments, the times can include each day of the week and specified times within each day presented in the form of a chart located on one face of the package wherein the days of the week are presented and the times within each day the medicaments are to be taken are presented in systematic fashion.

In addition, the kits can include instructional materials containing directions teaching the use of one or more NK1 receptor antagonists to reduce chronic consumption of a substance of abuse (e.g. alcohol) and/or to reduce cravings for such a substance. While the instructional materials typically comprise written or printed materials, they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

IV. Screening Systems.

This invention also contemplates methods of screening for agents that reduce/inhibit self-administration of a substance of abuse. It was a discovery of this invention that administration of NK1 receptor antagonists will inhibit self-administration of a substance of abuse (e.g. ethanol). Consequently it is believed that agents that inhibit expression or activity of an NK1 receptor or receptor pathway will reduce self-administration of a substance of abuse and/or cravings for such substance.

A wide variety of assays for antagonistic NK1 receptor activity are known to those of skill in the art. For example, one screening approach involves screening one or more test agents for the ability to inhibit the caudally directed, biting and scratching response elicited by intrathecal administration of the selective NK1 agonist Ac—[Arg⁶,Sar⁹,Met(O₂)¹¹]substance P6-11 in conscious mice (see, e.g., Iyengar et al. (1997) J. Pharmacol. Exp. Ther. 1 280(2): 774-785 for a description of the assay). In preferred test agents, the potentiation of the tail-flick response elicited by intrathecal administration of the NK1 agonist [Sar⁹,Met(O₂)¹¹]substance P in rats is also selectively blocked (see, e.g. Marusov et al. (1996) Eksp. Klin. Farmnakol., 59: 24-27; Iyengar et al. (1997) J. Pharmacol. Exp. Ther. 1 280(2): 774-785 for a description of the assay).

Another assay is a model of persistent nociceptive activation induced by tissue injury (the formalin test) (see, e.g., Henry et al. (1999) J. Neurosci. 19: 6588-6598; Iyengar et al. (1997) J. Pharmacol. Exp. Ther. 1 280(2): 774-785). The ability to block licking behavior in,this assay is an indication of NK1 antagonist activity.

Other assays for NK1 activity include, but are not limited to ex vivo binding activity. In such assays the ability of the putative antagonist (test agent) to inhibit binding of labeled substance P (e.g. ¹²⁵I-labeled substance P) to central and/or peripheral NK1 receptors provides an indication that such agents are potential NK1 antagonists. Methods of performing such binding assays are well known to those of skill in the art (see, e.g. Cascieri et al. (1995) Mol. Pharmacol., 47: 660-665).

In other assays, cells can be screened with test agents to identify agents that inhibit NK1 receptor expression and/or activity. The cells can be in cell culture, tissue preparations (e.g. brain slice preps) or animals. NK1 receptor expression can be assayed by detecting NK1 receptors and/or by detecting NK1 receptor nucleic acids (e.g. MRNAs). Activity can be assayed by screening activity of one or more components in an NK1 receptor pathway. Methods of detecting expression and/or activity of an NK1 receptor are well known to those of skill in the art as described above.

Once antagonistic NK1 activity is determined in one or more test agents, they can be further assayed for other adverse effects in mammals. Thus, for example, they can be assayed for neurological, motor, cardiovascular, gastrointestinal or autonomic side effects at appropriate dosages. Methods of evaluating such compounds are well known to those of skill in the art (see, e.g., Remington: The Science and Practice of Pharmacy, 19th Ed. (Easton, Pa.) Mack Publishing Co., 1995).

Because of the high cost of animals, in preferred embodiments, test agents are screened for the ability to antagonize NK1. Agents that antagonize NK1 are putative agents for inhibiting consumption of a substance of abuse.

Thus, for example, in certain embodiments, test agent(s) are screened for the ability to antagonize substance P signaling in a cell line expressing NK1 using, e.g., calcium release as the readout. For example, HEK cells expressing NK1 are loaded with a calcium sensitive dye. Then both substance P and test compounds (or vehicle) are added. Calcium release is measured by fluorescence or any other convenient method. This can be done in high throughput using a FLEX station. Agents identified as NK1 inhibitors in such an assay are putative agents for inhibiting consumption of a substance of abuse.

The agents that screen positive as NK1 antagonists in cell-based assays then can be subsequently screened in animal/behavioral studies, e.g. as described above.

### EXAMPLES

The following examples are offered to illustrate, but not to limit the claimed invention.

### Example 1

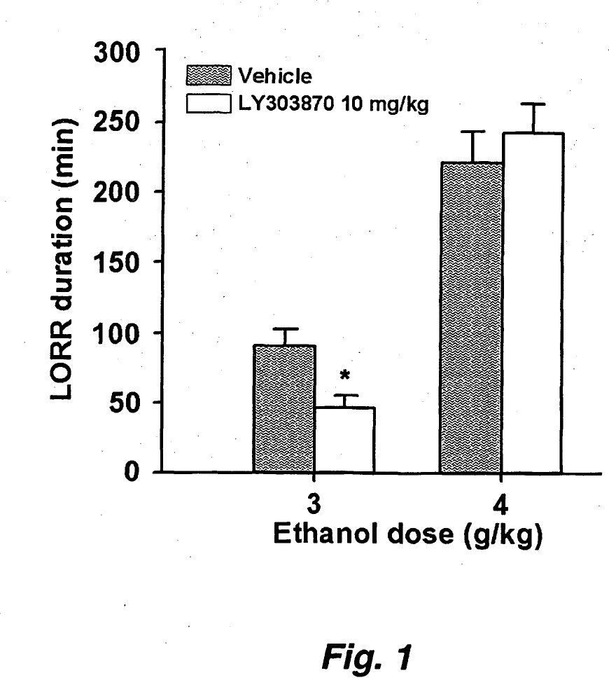

FIG. 1 shows the loss of righting reflex, a measure of intoxication. Ly303870 reduced the time the test animals were “drunk” when they were given 3 g/kg of ethanol.

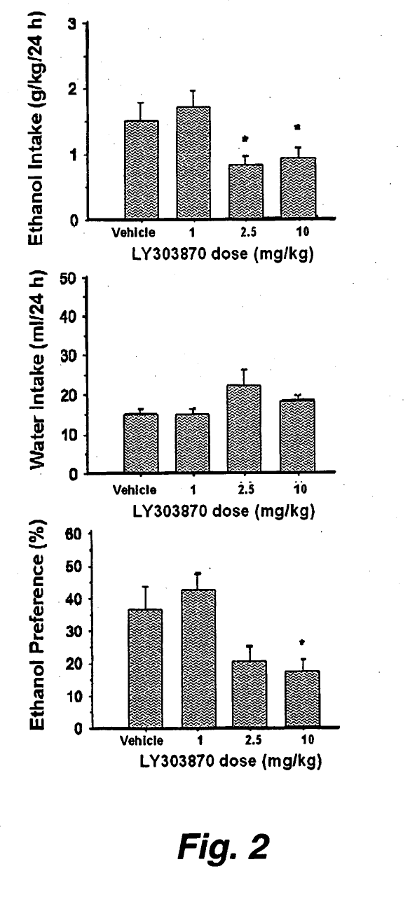

FIG. 2 illustrates the effect of administration of the NK1 receptor antagonist LY303870 on self-administration of alcohol by a rat. Concentrations of 2.5 mg/kg or greater resulted in a significant reduction in self-administration of ethanol. In addition, the data show a reduced preference for ethanol as compared to water in the treated animals indicating that reduced ethanol consumption did not simply reflect reduced drinking behavior.

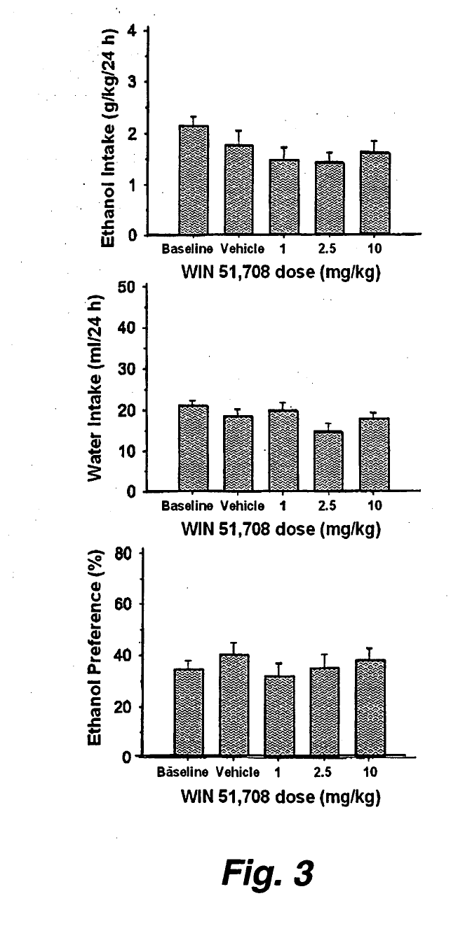

FIG. 3 shows that WIN 51,708 did not have a significant effect on ethanol consumption and did not alter preference. Without being bound to a particular theory, it is believed that this effect may be due to failure of WIN 51,708 to cross the blood-brain barrier.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of inhibiting or reducing self-administration of a substance of abuse by a mammal, said method comprising administering to said mammal an NK1 receptor antagonist in a concentration sufficient to reduce self-administration of a substance of abuse.

2. The method of claim 1, wherein said substance of abuse is selected from the group consisting of cocaine, an amphetamine, an opiate, a cannabinoid, nicotine, and alcohol.

3. The method of claim 1, wherein said substance of abuse is alcohol.

4. The method of claim 1, wherein said administering reduces preference for the substance of abuse.

5. The method of claim 4, wherein said administering reduces preference for alcohol.

6. The method of claim 1, wherein said NK1 receptor antagonist is selected from the group consisting of LY303870, Sigma WIN 51,708, GR205171A, Takeda TAK-637, SR-140333, Merc MK869, MEN 11467, triazole NK1 receptor antagonists (see WO 99/38533A1), CP-99,994, GlaxoSmith-Kline GW597599, and CJ-12,255

7. The method of claim 6, wherein said NK1 receptor antagonist is LY303870.

8. The method of claim 6, wherein said NK1 receptor antagonist is Sigma WIN 51,708.

9. The method of claim 1, wherein said NK1 receptor antagonist is orally administered.

10. The method of claim 1, wherein said NK1 receptor antagonist is injected.

11. The method of claim 1, wherein said NK1 receptor antagonist is in a unit dosage formulation.

12. The method of claim 1, wherein said mammal is a human.

13. The method of claim 1, wherein said mammal is a human.

14. The method of claim 1, wherein said mammal is a non-human mammal.

15. The method of claim 13, wherein said mammal shows one or more symptoms characteristic of addiction to a substance of abuse.

16. The method of claim 13, wherein said mammal is diagnosed as chronically consuming a substance of abuse.

17. The method of claim 13, wherein said mammal has ceased chronic consumption of a substance of abuse.

18. The method of any one of claims 15 through 17, wherein said substance of abuse is alcohol.

19. The method of claim 1, wherein said NK1 receptor antagonist is administered at a dosage ranging from about 1 to about 100 mg/kg daily.

20. The method of claim 1, wherein said NK1 receptor antagonist is administered in a unit dosage formulation.

21. A kit for inhibiting or reducing self-administration of a substance of abuse, said kit comprising:
a container containing one or more NK1 receptor antagonists; and
instructional materials teaching the use of an NK1 receptor antagonist to inhibit self-administration of a substance of abuse.

22. The kit of claim 21, wherein said NK1 receptor antagonist is selected from the group consisting of LY303870, Sigma WIN 51,708, GR205171A, Takeda TAK-637, SR-140333, Merc MK869, MEN 11467, triazole NK1 receptor antagonists (see WO 99/38533A1), CP-99,994, GlaxoSmith-Kline GW597599, and CJ-12,255

23. The kit of claim 21, wherein said NK1 receptor antagonist is LY303870.

24. The kit of claim 21, wherein said NK1 receptor antagonist is Sigma WIN 51,708.

25. The kit of claim 21, wherein said substance of abuse is alcohol.

26. A method of screening for an agent that reduces self-administration of a substance of abuse by a mammal, said method comprising:
administering a test agent to said mammal; and
screening said mammal for NK1 antagonistic activity, wherein NK1 antagonistic activity indicates that said agent is an agent that is likely to reduce self administration of a substance of abuse.

27. The method of claim 26, wherein said screening comprises screening said test agent for the ability to inhibit a caudally directed, biting and scratching response elicited by intrathecal administration of Ac—[Arg⁶,Sar⁹,Met(O₂)¹¹] substance P6-11 in conscious mice.

28. The method of claim 26, wherein said screening comprises screening said test agent for the ability to inhibit or fully block the potentiation of the tail-flick response elicited by intrathecal administration [Sar⁹,Met(O₂)₁₁]substance P in rats.

29. A method of screening for an agent that reduces self-administration of a substance of abuse by a mammal, said method comprising:
contacting a cell comprising an NK1 receptor with a test agent; and
detecting expression or activity of said NK1 receptor or an NK1 receptor pathway, wherein an inhibition of NK1 receptor expression or activity as compared to NK1 receptor expression or activity in a control cell indicates that said test agent is a candidate agent for use in reducing self-administration of a substance of abuse by a mammal.

30. The method of claim 29, wherein said cell is a neural cell.

31. The method of claim 29, wherein said cell is a neural cell in a brain slice preparation.

32. The method of claim 29, wherein said cell is a neural cell in in vitro culture.

33. The method of claim 29, wherein said cell is a HEK cell expressing an NK1 receptor.
